(19) United States
(12) Patent Application Publication
(10) Pub. No.: US 2026/0306538 A1
(43) Pub. Date: Oct. 8, 2026

(54) Application of CCR5hi Innate Immune Memory Cells in Preparation of Immunotherapeutic Drug for Treating Sepsis

(71) Applicant: Children’s Hospital of Soochow University, Suzhou (CN)

(72) Inventors: Huiting ZHOU, Suzhou (CN); Jian WANG, Suzhou (CN)

(21) Appl. No.: 19/441,933

(22) Filed: Jan. 7, 2026

Related U.S. Application Data
(63) Continuation of application No. PCT/CN2025/137377, filed on Nov. 25, 2025.

(30) Foreign Application Priority Data
Apr. 2, 2025 (CN) 202510406796.9

(51) Int. Cl. A61K 35/15; A61K 9/00; A61K 40/17; A61K 40/42; A61P 31/02; C07K 14/705; C07K 14/715; C12N 5/0786

(52) U.S. Cl. CPC A61K 35/15; A61K 9/0019; A61K 40/17; A61K 40/4219; A61P 31/02; C07K 14/70553; C07K 14/70589; C07K 14/7158; C12N 5/0645; A61K 2239/31

(57) ABSTRACT

An application of innate immune memory cells with high expression of C-C chemokine receptor type 5 (CCR5^{hi} innate immune memory cells) in preparation of an immunotherapeutic drug for treating sepsis is provided, relating to the technical field of biomedicines. It provides an innate immune memory cell with a protective effect against sepsis, which is induced by trained immunity via Bacillus Calmette-Guérin (BCG) combined with bacterial lipoprotein, namely, a CD45⁺CD11b⁺Ly6G⁻Ly6C⁺CCR5^{hi} monocyte. Through single-cell sequencing analysis and functional validation, the cell subset characteristics are precisely identified, and its functional mechanisms are elucidated, thereby addressing the key gap of unclear effector cell identity and undefined action targets. This provides a cellular-level theoretical basis and technical solution for the targeted immunotherapy of sepsis, which is expected to achieve better therapeutic outcomes in clinical practice.

### CROSS-REFERENCE TO RELATED APPLICATION

This application claims priority to Chinese patent application No. CN202510406796.9, filed to China National Intellectual Property Administration (CNIPA) on Apr. 2, 2025, which is herein incorporated by reference in its entirety.

### TECHNICAL FIELD

The disclosure relates to the technical field of biomedicines, and particularly to an application of innate immune memory cells with high expression of C-C chemokine receptor type 5 (CCR5^{hi} innate immune memory cells) in preparation of an immunotherapeutic drug for treating sepsis.

### BACKGROUND

Sepsis is a multiple organ dysfunction syndrome caused by a dysregulated host response to infection, accompanied by severe immune imbalance during its pathogenesis. The dysregulation of pro-inflammatory and anti-inflammatory mechanisms disrupts body homeostasis, leading to disease progression and even life-threatening conditions. Currently, the treatment of sepsis primarily relies on anti-infective therapies (such as antibiotics), fluid resuscitation, and organ support. However, the overuse of antibiotics has led to the emergence of multidrug-resistant strains, and even the rise of superbugs that are fully resistant to all existing antibiotics. This phenomenon presents a severe challenge to traditional anti-infective strategies and highlights the urgent need to develop innovative immunotherapeutic strategies to improve the prognosis of sepsis.

Trained immunity (TI) is an emerging immunomodulatory strategy, primarily relying on innate immune cells such as monocytes/macrophages, natural killer (NK) cells, and innate lymphoid cells (ILCs). These cells sense pathogen-associated molecular patterns (PAMPs) via pattern recognition receptors (PRRs), thereby inducing epigenetic and metabolic reprogramming. This enables them to mount a stronger immune response upon re-encounter with the same or different pathogen stimuli, thus accelerating pathogen clearance and restoring body homeostasis. The immune-enhancing effects of TI demonstrate broad application prospects in combating viral, bacterial, and fungal infections, showing significant potential particularly in the prevention and treatment of sepsis. Studies have shown that inducing TI can enhance the pro-inflammatory response and improve bacterial clearance capacity in septic mice, thereby increasing survival rates. Therefore, developing effective TI induction strategies is expected to overcome the limitations of traditional antibiotic therapy and provide new insights for the immunomodulation of sepsis.

Many biological agents have been confirmed to induce TI. Specifically, the attenuated live vaccine Bacillus Calmette-Guérin (BCG), widely used for tuberculosis prevention, is currently one of the most established and widely applied TI inducers. As a classic TI inducer, BCG drives metabolic and epigenetic reprogramming in innate immune cells via PRRs such as Toll-like receptors (TLRs), enhancing the host’s non-specific immune defense capabilities. In recent years, the immunomodulatory potential of BCG has gradually expanded to fields such as cancer, viral infections, and autoimmune diseases, and its role in sepsis prevention and treatment has also garnered significant attention. For example, neonatal BCG vaccination can promote emergency granulopoiesis, enabling effective control of early infections in sepsis.

Although BCG can enhance anti-infective capacity through TI, its application in sepsis treatment still has limitations as follows. 1. The inflammatory response induced by BCG may exacerbate immune imbalance in sepsis patients. 2. Its mechanism of action is not yet fully understood, particularly regarding which innate immune cells play a key role in the BCG-induced protection against sepsis. 3. Traditional BCG vaccination carries certain safety risks, especially in immunocompromised individuals.

Therefore, developing safer and more efficient TI induction strategies and clarifying the core effector cell types and their mechanisms of action are crucial for optimizing the immunomodulation of sepsis.

### SUMMARY

In view of deficiencies of the related art, an objective of the disclosure is to provide an application of innate immune memory cells with high expression of C-C chemokine receptor type 5 (CCR5^{hi} innate immune memory cells) in preparation of an immunotherapeutic drug for treating sepsis. The disclosure provides an innate immune memory cell with a protective effect against sepsis, which is induced by trained immunity via BCG combined with bacterial lipoprotein (BLP), namely, the CCR5^{hi} monocyte. Through single-cell sequencing analysis and functional validation, the cell subset characteristics are precisely identified, and its functional mechanisms are elucidated, thereby addressing the key gap in the related art of unclear effector cell identity and undefined action targets. This provides a cellular-level theoretical basis and technical solution for the targeted immunotherapy of sepsis.

To achieve the above objective, the disclosure adopts the following technical solutions.

The disclosure provides an application of CCR5^{hi} innate immune memory cells in preparation of an immunotherapeutic drug for treating sepsis.

In an embodiment, the CCR5^{hi} innate immune memory cells are a cell subset induced by trained immunity via BCG combined with BLP, co-expressing markers CD45⁺CD11b⁺Ly6G⁻Ly6C⁺CCR5^{hi} (i.e., cells that are positive for the leukocyte common antigen cluster of differentiation 45 abbreviated as CD45 and the myeloid marker cluster of differentiation 11b abbreviated as CD11b, negative for the neutrophil marker lymphocyte antigen 6 complex, locus G abbreviated as Ly6G, express high levels of the monocyte activation marker lymphocyte antigen 6 complex, locus C abbreviated as Ly6C, and exhibit high surface expression of the chemokine receptor CCR5).

In an embodiment, a dosage form of the immunotherapeutic drug is an injection.

In an embodiment, a route of administration of the immunotherapeutic drug includes intravenous injection, intraperitoneal injection, intramuscular injection, or subcutaneous injection.

In an embodiment, the immunotherapeutic drug further includes a pharmaceutically acceptable carrier.

In an embodiment, the carrier includes one or more selected from the group consisting of a disintegrant, a diluent, a lubricant, a binder, a wetting agent, a flavoring agent, a suspending agent, a surfactant, and a preservative.

Compared with the related art, the disclosure provides the following beneficial effects.
(1) The disclosure provides a cell subset with innate immune memory characteristics, CCR5^{hi} monocytes, induced by trained immunity via BCG combined with BLP. This cell subset exhibits enhanced antimicrobial activity and heightened inflammatory responsiveness, enabling rapid response upon the onset of sepsis to enhance the body’s immune defense capabilities, thereby significantly improving sepsis prognosis. The disclosure is expected to overcome the limitations of existing antibiotic therapies, provide a theoretical foundation for the precise regulation of trained immunity, and open new avenues for the immunotherapy of sepsis.
(2) To overcome the limitations of BCG-induced trained immunity in sepsis treatment, the disclosure proposes a strategy of optimizing trained immunity via BCG combined with BLP. BLP, as a TLR2-specific agonist, differs from the potent pro-inflammatory response mediated by LPS-TLR4. It promotes phagosome maturation in macrophages by upregulating Ras-related protein Rab-10 (Rab10) and activates the C-X-C motif chemokine ligand 2-C-X-C motif chemokine receptor 2 (CXCL2-CXCR2) pathway to recruit neutrophils, thereby bypassing the TLR4-dependent inflammatory pathway to directly enhance pathogen clearance, forming an immune protection mode characterized by low inflammation-high bactericidal activity. To gain deeper insights into the mechanisms of BCG+BLP-induced trained immunity, 10X Genomics single-cell sequencing is employed in the disclosure. A subset of CCR5^{hi} monocytes exhibiting features of innate immune memory is identified. This subset is rapidly activated upon sepsis onset, enhanced host immune defense is conferred, and sepsis prognosis is significantly improved. Prior to infection, CCR5^{hi} monocytes can be pre-activated to prime the innate immune system, enabling the host to release an appropriate amount of inflammatory cytokines promptly after pathogen exposure. As a result, recruitment of phagocytes and pathogen recognition are accelerated, allowing effective pathogen clearance before infection becomes uncontrolled. The vicious cycle of “inflammatory imbalance → tissue damage → secondary exacerbation of inflammation” is thereby interrupted. Ultimately, the survival rate of neonatal mice with sepsis is increased. Unlike conventional anti-inflammatory agents such as glucocorticoids, this strategy is not only associated with reduced inflammation-related adverse effects, but also enhanced early immune responses and improved pathogen clearance capacity are achieved.
(3) The disclosure provides new ideas and methods for the treatment of sepsis, which are expected to achieve better therapeutic outcomes in clinical practice. By precisely modulating CCR5^{hi} monocytes, the progression of sepsis can be more effectively controlled, sepsis mortality reduced, and patient prognosis improved, demonstrating significant clinical application value and broad market prospects.
(4) The disclosure clearly identifies CCR5^{hi} monocytes as the effector cell population, enabling more precise therapeutic intervention. This reduces impacts on non-relevant cells and tissues, lowers side effects and risks during treatment, improves treatment safety, and overcomes the insufficient understanding of treatment safety and efficacy in the related art.

### BRIEF DESCRIPTION OF DRAWINGS

FIGS. 1A-1C illustrate expression of CCR5 in a cluster 5 monocyte subset. Specifically, FIG. 1A illustrates a Kyoto Encyclopedia of Genes and Genomes (KEGG) enrichment analysis plot of 715 upregulated genes in the cluster 5 monocyte subset; FIG. 1B illustrates a t-distributed Stochastic Neighbor Embedding (t-SNE) plot showing an expression level of C-C chemokine receptor type 5 (Ccr5) gene in monocytes, where each dot represents a single cell and dot color represents an average expression level of the Ccr5 gene; and FIG. 1C illustrates a violin plot showing expression of Ccr5 across different monocyte subsets.

FIG. 2 illustrates a flow cytometry sorting scheme for CCR5^{hi} monocytes.

FIGS. 3A-3B illustrate bacterial phagocytic capacity of monocyte cells from different groups. Specifically, FIG. 3A illustrates Luria-Bertani (LB) agar plate colony images showing Escherichia coli (E. coli) phagocytosed by monocyte cells from different groups; and FIG. 3B illustrates a statistical bar chart of bacterial colonies phagocytosed by the monocyte cells from different groups (n=4), where CCR5^{lo} represents CD45⁺CD11b⁺Ly6G⁻Ly6C⁺CCR5^{lo} cells; and CCR5^{hi} represents CD45⁺CD11b⁺Ly6G⁻Ly6C⁺CCR5^{hi} cells.

FIGS. 4A-4B illustrate expression levels of inflammatory factors in supernatant of cells from different groups. Specifically, FIG. 4A illustrates interleukin-6 (IL-6) expression level in the cell supernatant after 12 hours of LPS stimulation (n=4); and FIG. 4B illustrates tumor necrosis factor-alpha (TNF-α) expression level in the cell supernatant after 12 hours of LPS stimulation (n=4), where CCR5^{lo} represents CD45⁺CD11b⁺Ly6G⁻Ly6C⁺CCR5^{lo} cells, CCR5^{hi} represents CD45⁺CD11b⁺Ly6G⁻Ly6C⁺CCR5^{hi}, * indicates P<0.05, ** indicates P<0.01.

FIGS. 5A-5C illustrate comparison of improvement effects on septic mice in different groups. Specifically, FIG. 5A illustrates survival curves of mice in different groups (n=10 or 12); FIG. 5B illustrates representative images of hematoxylin and eosin (HE) staining of mouse lung tissue, scale bar = 100 micrometers (µm); and FIG. 5C illustrates a statistical bar chart of pathological injury scores based on HE staining of lung tissue (n=4), where * indicates P<0.05, *** indicates P<0.001.

### DETAILED DESCRIPTION OF EMBODIMENTS

The disclosure is further described in detail below through specific illustrated embodiments, but the disclosure is not limited to the following embodiments.

It should be noted that, unless otherwise specified, chemical reagents involved in the disclosure are purchased from commercial sources.

The BCG strain used in the disclosure was donated by the Suzhou Center for Disease Control and Prevention, China, and the Escherichia coli strain used was ATCC 8099.

### Embodiment 1

### Isolation of the CCR5^{hi} Cell Subsets

### 1. Establishment of the Trained Immunity Model

C57BL/6 neonatal mice are randomly divided into four groups, with 10 mice in each group. BCG and BLP are selected as trained immunity inducers. The body weight of 7-day-old mice is measured, and BCG (250 micrograms per gram abbreviated as μg/g body weight) and BLP (5 μg/g body weight) are administered via intraperitoneal injection to induce trained immunity. After injection, the mice are placed in an appropriate breeding environment and continued to be nursed by their dams for 3 days

### 2. Preparation of Cecal Slurry (CS)

Five male and female C57BL/6 mice aged 8 weeks are selected and anesthetized with pentobarbital (3%, 1 microliter per gram abbreviated as μL/g body weight). The cecal tissue is immersed in 75% ethanol and then processed in a biosafety cabinet. Using sterile scissors and forceps, the cecum is placed into a sterile petri dish. A 50 milliliters (mL) centrifuge tube is pre-weighed (weight M1). One end of the cecum is cut open with scissors, and the cecal contents are collected into the centrifuge tube using sterile forceps. The tube is re-weighed (weight M2), and the difference (M2-M1) represents the weight of the cecal contents. The centrifuge tube containing the cecal contents is supplemented with an appropriate volume of sterile 0.5% glucose saline to achieve a final concentration of 100 milligrams per milliliter (mg/mL). The mixture is thoroughly vortexed and sequentially filtered through 100 μm and 70 μm filters to remove undissolved residues. The cecal slurry is then distributed into aliquots while vortexing to ensure consistent concentration across tubes. Finally, the prepared cecal slurry is stored at -80℃ and used within 3 months.

### 3. Establishment of the Sepsis Model

A polymicrobial sepsis model in neonatal mice is established by intraperitoneal injection of CS. Specific operations are as follows. CS (100 mg/mL) is taken from the -80℃ freezer, thawed, and thoroughly vortexed. Subsequently, the body weight of 10-day-old mice is measured, and CS is injected using an insulin syringe at a dose of 1.0 mg/g body weight. One hour, 12 hours, and 24 hours after sepsis modeling, mice are anesthetized with pentobarbital (3%, 1 μL/g body weight) and sacrificed for sample collection for subsequent experiments. Survival status, health condition, and body weight changes are monitored continuously over 8 consecutive days.

### 4. Isolation of Peripheral Blood Mononuclear Cells (PBMCs)

After modeling, mice are anesthetized, and blood is collected from the heart using an insulin syringe. Whole blood is collected into ethylenediaminetetraacetic acid (EDTA)-containing anticoagulant tubes and diluted with an equal volume of PBS. In a 15 mL centrifuge tube, 3 mL of separation medium is added, and the diluted whole blood is carefully layered on top of the separation medium using a pipette to form a gradient. The tube is then centrifuged at 500 g for 30 minutes at room temperature, with the centrifuge deceleration set to 1. After centrifugation, distinct layers are observed. The mononuclear cell layer is gently aspirated using a pipette and transferred into a new 15 mL centrifuge tube. This fraction is defined as PBMCs.

### 5. Identification of CCR5^{hi} Monocyte Subsets by Single-Cell Sequencing

The PBMCs isolated in the previous step are subjected to single-cell sequencing. Cell subsets are annotated through clustering analysis, resulting in the identification of eight major cell types: monocytes (CD14⁺, Csf1r⁺, Lyz2⁺), B lymphocytes (CD79a⁺, Ms4a1⁺, CD19⁺), T lymphocytes (Cd3d⁺, Cd3e⁺, CD3g⁺), neutrophils (Ngp⁺, S100a8⁺), dendritic cells (Cd74⁺, Itgax⁺), macrophages (Ppbb⁺), red blood cells (Hba-a1⁺, Hba-a2⁺), and NK cells (Nkg7⁺, Klrblc⁺). Monocytes are further subdivided by re-clustering, and cluster 5 is found to be significantly expanded in BCG+BLP-induced trained immunity.

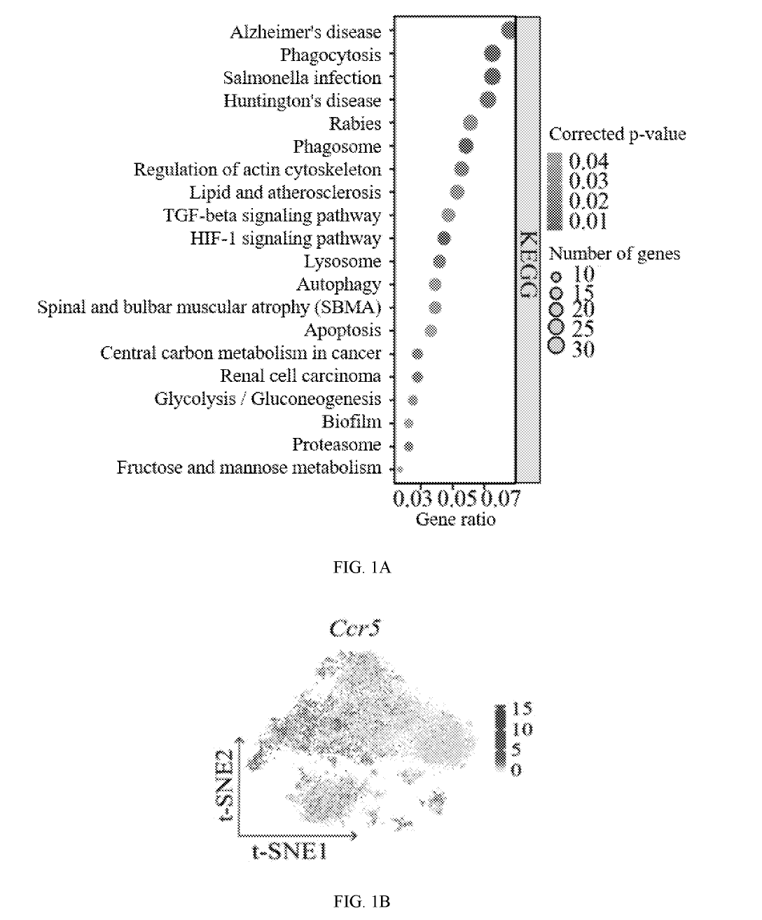

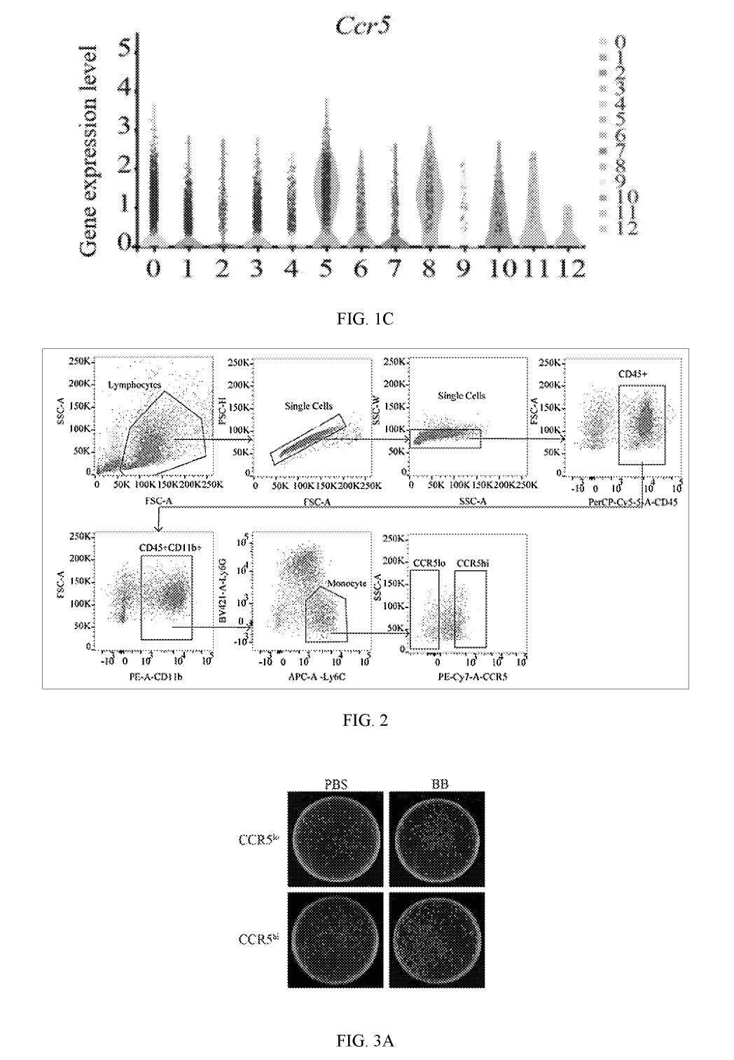

As can be seen from FIG. 1 A, KEGG enrichment analysis reveals that the 715 differentially expressed genes upregulated in cluster 5 are biologically associated with endocytosis. Phagocytosis is a special form of endocytosis. As can be seen from FIG. 1B, it can be seen that CCR5, a gene related to phagocytic function, is significantly upregulated in the cluster 5 monocyte subset compared to other monocytes. As can be seen from FIG. 1C, the violin plot further shows that CCR5 is expressed in all monocyte populations, but it is observed to be particularly abundant in the cluster 5 monocyte subset, proving that CCR5^{hi} monocytes are an innate immune memory subset mediated by BCG+BLP.

### 6. Flow cytometry detection of CCR5^{hi} monocytes

a. A portion of the mouse PBMC suspension prepared by density gradient centrifugation is taken and adjusted to a cell concentration of approximately 1×10⁶ cells/mL.
b. 50 µL of the cell suspension is mixed with 50 μL of fluorescence-activated cell sorting (FACS) buffer pre-mixed with antibodies targeting CD11b, Ly-6G, Ly-6C, and CCR5. The final volume ratio of cell suspension to antibody stock solution is maintained at 100:1. The mixture is incubated on ice in the dark for 30 minutes to avoid photobleaching.
c. After antibody incubation, 300 µL of FACS buffer is added to each tube, followed by centrifugation at 500 g, 4℃ for 5 minutes.
d. The supernatant is discarded, and 300 µL of FACS buffer is added to each tube to resuspend the cell pellet. The resuspended cells are thoroughly vortexed and transferred into flow cytometry tubes. Detection is performed using a flow cytometer, and data analysis is carried out with FlowJo software. The flow cytometry sorting scheme is shown in FIG. 2. The relevant steps are as follows.
   (1) Based on parameters of forward scatter area (FSC-A) and side scatter area (SSC-A), a viable cell gate is set to exclude debris and dead cells.
   (2) Doublets are excluded by gating on FSC-A vs forward scatter height (FSC-H) and SSC-A vs side scatter width (SSC-W) to ensure analysis of single cells.
   (3) CD45⁺ leukocytes are identified by staining with anti-CD45 antibody.
   (4) Within the CD45⁺ leukocytes, a myeloid cell subset is gated based on positive expression of CD11b.
   (5) Monocyte subsets are specifically isolated from the myeloid cell subset by dual staining with anti-Ly6G and anti-Ly6C antibodies, based on the characteristic surface marker combination LY6G⁻/LY6C⁺.
   (6) Within the monocyte subset, the CCR5^{hi} monocyte subset is finally gated based on the intensity of CCR5 expression.

### Embodiment 2

Antimicrobial Activity and Inflammatory Response Capability of CCR5^{hi} Monocytes

A control group (PBS) and a trained immunity group (BB) are designed for this experiment. The control group (PBS) is administered 50 μL of PBS via intraperitoneal injection in mice, while the trained immunity group (BB) is administered BCG (250 μg/g body weight) and BLP (5 μg/g body weight) by intraperitoneal injection. Using flow cytometry, CD45⁺CD11b⁺Ly6G⁻Ly6C⁺CCR5^{lo} cells and CD45⁺CD11b⁺Ly6G⁻Ly6C⁺CCR5^{hi} cells are isolated from the bone marrow of mice in both groups. These cells are divided into four subgroups: PBS-CCR5^{lo}, PBS-CCR5^{hi}, BB-CCR5^{lo}, and BB-CCR5^{hi}. Subsequently, the cells are seeded into cell culture plates at a density of 1×10⁵ cells per well. The cells are then infected with E. coli or stimulated with LPS, followed by collection of both cells and supernatants for functional assays to evaluate their phagocytic activity and inflammatory response capacity.

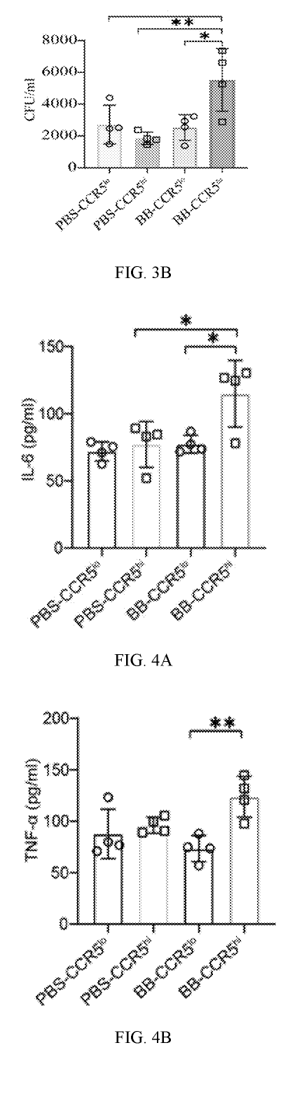

The four groups of sorted monocytes are co-incubated with E . coli in bacterial culture dishes for 30 minutes. The number of monocytes is set at 1×10⁵ cells, and E. coli is used at a concentration of 8×10⁶ CFU. Phagocytic activity of CCR5^{lo} and CCR5^{hi} monocytes is quantified using a serial dilution plating method, and the results are shown in FIGS. 3A-3B. As shown in FIGS. 3A-3B, compared to the control group CCR5^{lo} and CCR5^{hi} monocytes, BB-CCR5^{hi} monocytes exhibit significantly increased phagocytosis of E. coli. In comparison with BB-CCR5^{lo} monocytes, BB-CCR5^{hi} monocytes also show markedly enhanced phagocytic ability. Furthermore, compared to PBS-CCR5^{lo} and PBS-CCR5^{hi} monocytes, BB-CCR5^{hi} monocytes show no statistically significant difference in bacterial phagocytosis.

After stimulation with LPS (100 ng/mL) for 12 hours, supernatants from the four monocyte groups are collected. The levels of inflammatory cytokines in the supernatants of PBS-CCR5^{lo}, PBS-CCR5^{hi}, BB-CCR5^{lo}, and BB-CCR5^{hi} cells are detected using enzyme-linked immunosorbent assay (ELISA). The results are shown in FIGS. 4A-4B. As can be seen from FIG. 4A, compared with PBS-CCR5^{hi}, BB-CCR5^{hi} monocytes secrete significantly higher levels of IL-6; and compared with BB-CCR5^{lo}, BB-CCR5^{hi} monocytes also secrete significantly higher levels of IL-6. Furthermore, as can be seen from FIG. 4B, compared with BB-CCR5^{lo}, BB-CCR5^{hi} monocytes secrete significantly higher levels of TNF-α. However, the TNF-α level released by BB-CCR5^{hi} monocytes is higher than that of PBS-CCR5^{hi} monocytes, but the difference does not reach statistical significance.

The above results indicate that the antimicrobial activity and inflammatory response capability of BB-CCR5^{hi} monocytes are both enhanced.

### Embodiment 3

Validation of the Effect of CCR5^{hi} Monocytes on Neonatal Mouse Sepsis by Adoptive Transfer

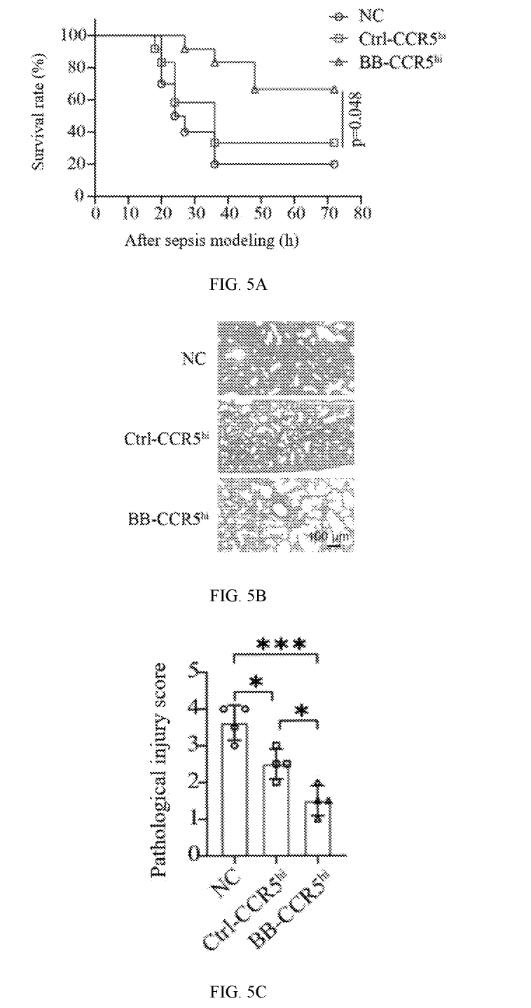

CD45⁺CD11b⁺Ly6G⁻Ly6C⁺CCR5^{hi} cells are isolated from the bone marrow of control (PBS) and trained immunity (BB) groups of mice. These cells are then injected via tail vein into recipient neonatal mice, forming three experimental groups: intravenous injection of PBS (NC group), intravenous injection of control CCR5^{hi} monocytes (Ctrl-CCR5^{hi} group), and intravenous injection of trained immunity CCR5^{hi} monocytes (BB-CCR5^{hi} group). The injection volume is 100 μL, and the cell number is 1×10⁶ per mouse. Subsequently, a polymicrobial sepsis model is induced in all mice. Samples are collected for detection of relevant indicators, and survival outcomes are monitored across the three groups. The results are shown in FIGS. 5A-5C.

As can be seen from FIG. 5A, in the NC group, 8 out of 10 mice die within 36 hours after sepsis induction, with a survival rate of 20% (2/10) at 72 hours; in the Ctrl-CCR5^{hi} group, 8 out of 12 mice die within 36 hours, with a survival rate of 33.3% (4/12) at 72 hours; in the BB-CCR5^{hi} group, only 4 out of 12 mice die within 48 hours, with a survival rate of 66.7% (8/12) at 72 hours. Compared to the Ctrl-CCR5^{hi} group, the survival rate of the BB-CCR5^{hi} group is significantly improved (P=0.048).

Lung tissues from mice 24 hours post-sepsis induction are collected and subjected to HE staining. The results are shown in FIGS. 5B and 5C. Compared to the NC group, lung inflammation and alveolar damage are significantly ameliorated in both the Ctrl-CCR5^{hi} and BB-CCR5^{hi} groups. Furthermore, compared to the Ctrl-CCR5^{hi} group, the degree of inflammatory cell infiltration and tissue injury in the lungs of the BB-CCR5^{hi} group is markedly reduced.

Finally, it should be noted that the above embodiments do not limit the disclosure in any form. For those skilled in the art, various modifications and improvements can be made on the basis of the disclosure. Therefore, any modifications or improvements made without departing from the spirit of the disclosure shall fall within the scope of protection claimed by the disclosure.

What is claimed is:

1. An application of innate immune memory cells with high expression of C-C chemokine receptor type 5 (CCR5^{hi} innate immune memory cells) in preparation of an immunotherapeutic drug for treating sepsis, wherein the CCR5^{hi} innate immune memory cells are a cell subset co-expressing CD45⁺CD11b⁺Ly6G⁻Ly6C⁺CCR5^{hi} induced by trained immunity in mice via Bacillus Calmette-Guérin (BCG) combined with bacterial lipoprotein (BLP), the CCR5^{hi} innate immune memory cells are configured to prepare the immunotherapeutic drug for treating septic mice.

2. The application as claimed in claim 1, wherein a dosage form of the immunotherapeutic drug is an injection.

3. The application as claimed in claim 1, wherein a route of administration of the immunotherapeutic drug comprises intravenous injection, intraperitoneal injection, intramuscular injection, or subcutaneous injection.

4. The application as claimed in claim 1, wherein the immunotherapeutic drug further comprises a pharmaceutically acceptable carrier.

5. The application as claimed in claim 4, wherein the carrier comprises one or more selected from the group consisting of a disintegrant, a diluent, a lubricant, a binder, a wetting agent, a flavoring agent, a suspending agent, a surfactant, and a preservative.
